(19) United States
(12) Patent Application Publication
(10) Pub. No.: US 2023/0094935 A1
(43) Pub. Date: Mar. 30, 2023

(54) METHOD OF MAKING TEREPHTHALIC ACID

(71) Applicant: SABIC Global Technologies B.V., Bergen op Zoom (NL)

(72) Inventors: Syed Azhar Hashmi, Riyadh (SA); Prasanna Kumar Pathath, Jubail (SA); Suman Kumar Jana, Bangalore (IN); Vadlamani Chaitanya, Yanbu Industrial City (SA); Naveen Sathu, Bangalore (IN); Lee Sangjoo, Yanbu Industrial City (SA); Debasish Das, Bangalore (IN); Selvakumar Subramanian, Bangalore (IN)

(21) Appl. No.: 17/802,865

(22) PCT Filed: Feb. 24, 2021
(86) PCT No.: PCT/IB2021/051552

§ 371 Date: Aug. 26, 2022

Related U.S. Application Data
(60) Provisional application No. 62/983,531, filed on Feb. 28, 2020.

(51) Int. Cl. C07C 51/265; B01J 27/128; B01J 35/12

(52) U.S. Cl. CPC C07C 51/265; B01J 27/128; B01J 35/12; C07C 2601/16

(57) ABSTRACT

Disclosed is a process for producing terephthalic acid. The process includes contacting p-xylene with a gaseous stream containing oxygen (O₂) in presence of a homogeneous catalyst solution, at a reaction temperature of 180° C. to 195° C. to oxidize at least a portion of the p-xylene and form a product stream containing terephthalic acid, said homogeneous catalyst solution contains 350 ppm to 450 ppm cobalt (Co), 170 ppm to 270 ppm manganese (Mn), and 410 ppm to 510 ppm bromine (Br), wherein a Br/(Co+Mn) wt. % ratio is 0.5:1 to 1:1, and a Co to Mn wt. % ratio is 1.5:1 to 2:1.

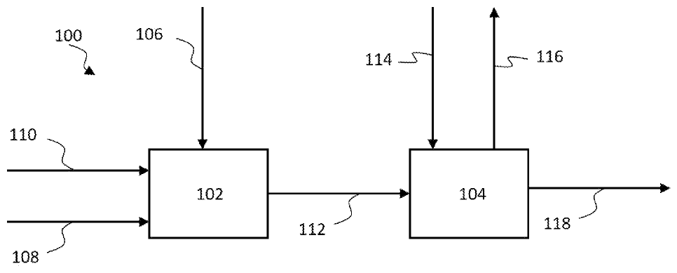

### CROSS REFERENCE TO RELATED APPLICATION

The present application is an international filing which claims priority to U.S. Provisional No. 62/983,531, filed Feb. 28, 2020, which is incorporated herein in its entirety.

### FIELD OF THE DISCLOSURE

The present disclosure generally relates to a method of making terephthalic acid using a catalyst (e.g., a CMB catalyst) comprising cobalt (Co), manganese (Mn), and bromine (Br).

### BACKGROUND OF THE DISCLOSURE

Terephthalic acid is a commodity chemical and can be used as raw material for various industrial processes. For example, terephthalic acid is a precursor to polyethylene terephthalate (PET), which can be used in clothing materials and plastic bottles. Some current commercially available processes for producing terephthalic acid typically rely on the oxidation of p-xylene with oxygen. However, such processes tend to produce unwanted impurities such as 4-carboxybenzaldehyde (4-CBA), p-toluic acid, and/or other colored impurities.

While attempts have been made to produce more efficient CMB catalysts, these attempts have the potential to create additional problems and may not solve the problems associated with the production of the aforementioned unwanted impurities. For example, Japanese Publication JP2017095391 discloses a process for producing terephthalic acid with a CMB catalyst composition that can contain upwards of 10,000 parts per million by weight (ppm) of Co and Mn. One of the potential issues with such a catalyst is that high amounts of Co and Mn can negatively impact the production economics for terephthalic acid. In another example, U.S. Pat. No. 4,051,178 discloses the use of a CMB catalyst that preferably uses 700 ppm to 1500 ppm of Br. Br, however, can be corrosive, especially for the equipment used in the terephthalic acid production process.

Some current CMB catalysts can be economically inefficient for large scale terephthalic acid production, can be corrosive to the equipment used in such processes, and/or can lead to unwanted production of impurities such as 4-CBA, p-toluic acid, and/or other colored impurities. These and other inefficiencies and opportunities for improvement are addressed and/or overcome by the systems, methods and processes of the present disclosure.

### SUMMARY OF THE DISCLOSURE

The present disclosure provides that advantageous discoveries have been made that provide answers to at least some of the aforementioned problems associated with terephthalic acid production processes. In an aspect, an improved terephthalic acid production process can include the use of a CMB catalyst composition that can have relatively low amounts of Co, Mn, and Br. Further, the use of the CMB catalyst in terephthalic acid production can result in reduced impurity levels in the terephthalic acid product. In an embodiment, it was discovered that a CMB catalyst having 350 ppm to 450 ppm Co, 170 ppm to 270 ppm Mn, and 410 ppm to 510 ppm Br can result in reduced 4-CBA levels in the terephthalic acid product. These reduced levels of 4-CBA are advantageous in that they can lead to a reduction in the production costs associated with terephthalic acid and subsequently, PET.

In an aspect of the present disclosure, an improved process for producing terephthalic acid is described. The process can include contacting p-xylene with a gaseous stream comprising oxygen (O₂) in the presence of a homogeneous catalyst solution, at a reaction temperature of 180° C. to 195° C. to oxidize at least a portion of the p-xylene and form a product stream comprising terephthalic acid, the homogeneous catalyst solution comprising 350 ppm to 450 ppm cobalt (Co), 170 ppm to 270 ppm manganese (Mn), and 410 ppm to 510 ppm bromine (Br). In some aspects, the Br to (Co+Mn) weight % ratio (e.g., Br/(Co+Mn) wt. % ratio) in the homogeneous catalyst solution can be 0.5:1 to 1:1. In some aspects, the Co to Mn wt. % ratio in the homogeneous catalyst solution can be 1.5:1 to 2:1. In some aspects, the homogeneous catalyst solution comprises 390 ppm to 410 ppm of Co, 210 ppm to 230 ppm of Mn, and 450 ppm to 470 ppm of Br. In some aspects, the Br to (Co+Mn) wt. % ratio in the homogeneous catalyst solution can be 0.6:1 to 0.9:1. In some aspects, the Co to Mn wt. % ratio in the homogeneous catalyst solution can be 1.75:1 to 1.85:1. In some aspects, the Co to Br wt. % ratio in the homogeneous catalyst solution can be 0.8:1 to 0.95:1. In some aspects, the Mn to Br wt. % ratio in the homogeneous catalyst solution can be 0.4:1 to 0.55:1. In some aspects, the reaction temperature can be 187° C. to 191° C. In some aspects, the product stream can include less than 0.3 weight % (wt. %) of 4-CBA, based on the total weight of the product stream. The homogeneous catalyst solution can include an acid. In some particular aspects, the acid can be acetic acid. In some aspects, the gaseous stream can include 19 volume % (vol. %) to 25 vol. % of O₂, based on the total volume of the gaseous stream. In some aspects, the gaseous stream can include air. In some aspects, the p-xylene can be contacted with the gaseous stream at a reaction pressure 10 bar to 14 bar (1,000 to 1,400 kilopascals). In some aspects, the homogeneous catalyst solution can be obtained by contacting p-xylene with a solution containing 3 wt. % to 7 wt. % of Co, 1 wt. % to 5 wt. % of Mn and 12 wt. % to 18 wt. % of Br. The p-xylene in the homogenous catalyst solution can be contacted with the gaseous stream containing O₂ in a reactor and the homogeneous catalyst solution can be fed to the reactor at a flow rate of 70 kilograms per hour (Kg/hr) to 90 Kg/hr. In some aspects, the reactor can be a titanium lined reactor. In some aspects, the average residence time of the homogeneous catalyst solution in the reactor can be 0.5 hours to 2 hours. In some aspects, the CO₂ vol. % in a gaseous outlet stream of the reactor can be less than 1.1 vol. %, based on the total volume of the gaseous outlet stream. In some aspects, the p-xylene and O₂ can be fed to the reactor at a mole ratio 1:3 to 1:5. In some aspects, the amount of each of iron, sodium, copper, and/or nickel in the homogeneous catalyst solution can be less than 10 ppm, less than 5 ppm, or less than 1 ppm. In some aspects, the p-xylene conversion obtained can be 95% to 100%. In some aspects, the terephthalic acid yield obtained can be 90% to 100%.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component. The term “ppm” refer to parts per million by weight, based on the total weight, of material that includes the component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The phrase “and/or” can include “and” or “or.” To illustrate, A, B, and/or C can include: A alone, B alone, C alone, a combination of A and B, a combination of A and C, a combination of B and C, or a combination of A, B, and C.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present disclosure can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

Other objects, features and advantages of the present disclosure will become apparent from the following figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the scope of the disclosure will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein. Any combination or permutation of embodiments is envisioned. Additional advantageous features, functions and applications of the disclosed systems, methods and processes of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

For a more complete understanding, reference is now made to the following descriptions taken in conjunction with the accompanying drawings. Exemplary embodiments of the present disclosure are further described with reference to the appended figures. It is to be noted that the various steps, features and combinations of steps/features described below and illustrated in the figures can be arranged and organized differently to result in embodiments which are still within the scope of the present disclosure. To assist those of ordinary skill in the art in making and using the disclosed systems, methods and assemblies, reference is made to the appended figures, wherein:

The FIGURE is a schematic of an example process of the present disclosure to produce terephthalic acid.

### DETAILED DESCRIPTION OF THE DISCLOSURE

The present disclosure provides that advantageous discoveries have been made that provide answers to at least some of the aforementioned problems associated with terephthalic acid production processes. In an aspect, an improved process of the present disclosure can include oxidizing p-xylene using a CMB catalyst composition comprising 350 ppm to 450 ppm of cobalt (Co), 170 ppm to 270 ppm of manganese (Mn), and 410 ppm to 510 ppm of bromine (Br). An exemplary CMB catalyst composition of the present disclosure includes relatively low amounts of corrosive Br or bromide, and can be used to oxidize p-xylene at a relatively low temperature. Relatively low specific consumption of p-xylene and acetic acid can be obtained with the compositions and methods of the present disclosure. Further, relatively lower amounts of side products such as 4-carboxybenzaldehyde and/or p-toluic acid can be obtained with the processes and/or methods of the present disclosure.

These and other non-limiting aspects of the present disclosure are discussed in further detail in the following sections with reference to the FIGURE. The units shown in the FIGURE can include one or more heating and/or cooling devices (e.g., insulation, electrical heaters, jacketed heat exchangers in the wall) or controllers (e.g., computers, flow valves, automated values, etc.) that can be used to control temperatures and/or pressures of the processes. While only one unit is usually shown, it should be understood that multiple units can be housed in one unit.

Although embodiments of the present disclosure have been described with reference to blocks of the FIGURE, it should be appreciated that operation of the present disclosure is not limited to the particular blocks and/or the particular order of the blocks illustrated in the FIGURE. Accordingly, embodiments of the disclosure may provide functionality as described herein using various blocks in a sequence different than that of the FIGURE.

Referring to the FIGURE, systems, processes and methods for producing terephthalic acid according to examples of the present disclosure are described. The system 100 can include a feed mix drum 102 and an oxidation reactor 104. A first solution 106 including Co, Mn, and Br, and a second solution 108 including p-xylene, and a third solution 110 including acetic acid can be fed to the feed mix drum 102. The solutions 106, 108 and 110 can be fed to the feed mix drum 102 separately, or can mixed with each other at any combination (e.g., 106 and 108, or 106 and 110, or 108 and 110, or 106, 108 and 110) or any order and can be fed to the feed mix drum 102 as combined feed. A homogeneous catalyst solution can be obtained in the feed mix drum 102.

The homogeneous catalyst solution obtained in the feed mix drum 102 can include: (1) 350 ppm to 450 ppm or at least any one of, equal to any one of, or between any two of 350 ppm, 352 ppm, 354 ppm, 356 ppm, 358 ppm, 360 ppm, 362 ppm, 364 ppm, 366 ppm, 368 ppm, 370 ppm, 372 ppm, 374 ppm, 376 ppm, 378 ppm, 380 ppm, 382 ppm, 384 ppm, 386 ppm, 388 ppm, 390 ppm, 391 ppm, 392 ppm, 393 ppm, 394 ppm, 395 ppm, 396 ppm, 397 ppm, 398 ppm, 399 ppm, 400 ppm, 401 ppm, 402 ppm, 403 ppm, 404 ppm, 405 ppm, 406 ppm, 407 ppm, 408 ppm, 409 ppm, 410 ppm, 412 ppm, 414 ppm, 416 ppm, 418 ppm, 420 ppm, 422 ppm, 424 ppm, 426 ppm, 428 ppm, 430 ppm, 432 ppm, 434 ppm, 436 ppm, 438 ppm, 440 ppm, 442 ppm, 444 ppm, 446 ppm, 448 ppm, and 450 ppm of cobalt (Co); (2) 170 ppm to 270 ppm or at least any one of, equal to any one of, or between any two of 170 ppm, 172 ppm, 174 ppm, 176 ppm, 178 ppm, 180 ppm, 182 ppm, 184 ppm, 186 ppm, 188 ppm, 190 ppm, 192 ppm, 194 ppm, 196 ppm, 198 ppm, 200 ppm, 202 ppm, 204 ppm, 206 ppm, 208 ppm, 210 ppm, 211 ppm, 212 ppm, 213 ppm, 214 ppm, 215 ppm, 216 ppm, 217 ppm, 218 ppm, 219 ppm, 220 ppm, 221 ppm, 222 ppm, 223 ppm, 224 ppm, 225 ppm, 226 ppm, 227 ppm, 228 ppm, 229 ppm, 230 ppm, 232 ppm, 234 ppm, 236 ppm, 238 ppm, 240 ppm, 242 ppm, 244 ppm, 246 ppm, 248 ppm, 250 ppm, 252 ppm, 254 ppm, 256 ppm, 258 ppm, 260 ppm, 262 ppm, 264 ppm, 266 ppm, 268 ppm, and 270 ppm of manganese (Mn); and/or (3) 410 ppm to 510 ppm or at least any one of, equal to any one of, or between any two of 410 ppm, 412 ppm, 414 ppm, 416 ppm, 418 ppm, 420 ppm, 422 ppm, 424 ppm, 426 ppm, 428 ppm, 430 ppm, 432 ppm, 434 ppm, 436 ppm, 438 ppm, 440 ppm, 442 ppm, 444 ppm, 446 ppm, 448 ppm, 450 ppm, 451 ppm, 452 ppm, 453 ppm, 454 ppm, 455 ppm, 456 ppm, 457 ppm, 458 ppm, 459 ppm, 460 ppm, 461 ppm, 462 ppm, 463 ppm, 464 ppm, 465 ppm, 466 ppm, 467 ppm, 468 ppm, 469 ppm, 470 ppm, 472 ppm, 474 ppm, 476 ppm, 478 ppm, 490 ppm, 492 ppm, 494 ppm, 496 ppm, 498 ppm, 500 ppm, 502 ppm, 504 ppm, 506 ppm, 508 ppm, and 510 ppm, of bromine (Br). In some aspects, the total amount of Co and Mn in the homogeneous catalyst solution can be 500 ppm to 750 ppm, or at least any one of, equal to any one of, or between any two of 500 ppm, 505 ppm, 510 ppm, 515 ppm, 520 ppm, 525 ppm, 530 ppm, 535 ppm, 540 ppm, 545 ppm, 550 ppm, 555 ppm, 560 ppm, 565 ppm, 570 ppm, 575 ppm, 580 ppm, 585 ppm, 590 ppm, 595 ppm, 600 ppm, 605 ppm, 610 ppm, 615 ppm, 620 ppm, 625 ppm, 630 ppm, 635 ppm, 640 ppm, 645 ppm, 650 ppm, 655 ppm, 660 ppm, 665 ppm, 670 ppm, 675 ppm, 680 ppm, 685 ppm, 690 ppm, 695 ppm, 700 ppm, 705 ppm, 710 ppm, 715 ppm, 720 ppm, 725 ppm, 730 ppm, 735 ppm, 740 ppm, 745 ppm, and 750 ppm. The homogeneous catalyst solution can include a dissolved Co compound e.g., salt. Co in the homogeneous catalyst solution can be present as a Co ion. At least a portion of the Co ion can be Co⁺² . The homogeneous catalyst solution can include a dissolved Mn compound e.g., salt. Mn in the homogeneous catalyst solution can be present as a Mn ion. At least a portion of the Mn ion can be Mn⁺². The homogeneous catalyst solution can include a dissolved Br compound e.g., salt. At least a portion of Br in the homogeneous solution can be present as bromide ion (Br−). The Br to (Co+Mn) wt. % ratio in the homogeneous catalyst solution can be 0.5:1 to 1:1, or at least any one of, equal to any one of, or between any two of 0.5:1, 0.55:1, 0.6:1, 0.62:1, 0.64:1, 0.65:1, 0.66:1, 0.68:1, 0.7:1, 0.72:1, 0.74:1, 0.75:1, 0.76:1, 0.78:1, 0.8:1, 0.82:1, 0.84:1, 0.85:1, 0.86:1, 0.88:1, 0.9:1, 0.95:1 and 1:1. The Co to Mn wt. % ratio in the homogeneous catalyst solution can be 1.5:1 to 2:1, or at least any one of, equal to any one of, or between any two of 1.5:1, 1.55:1, 1.6:1, 1.65:1, 1.7:1, 1.71:1, 1.72:1, 1.73:1, 1.74:1, 1.75:1, 1.76:1, 1.77:1, 1.78:1, 1.79:1, 1.8:1, 1.81:1, 1.82:1, 1.83:1, 1.84:1, 1.85:1, 1.86:1, 1.87:1, 1.88:1, 1.89:1, 1.9:1, 1.95:1 and 2:1. In some aspects, the Co to Br wt. % ratio in the homogeneous catalyst solution can be 0.8:1 to 0.95:1, or at least any one of, equal to any one of, or between any two of 0.8:1, 0.81:1, 0.82:1, 0.83:1, 0.84:1, 0.85:1, 0.86:1, 0.87:1, 0.88:1, 0.89:1, 0.9:1, 0.91:1, 0.92:1, 0.93:1, 0.94:1, and 0.95:1. In some aspects, the Mn to Br wt. % ratio in the homogeneous catalyst solution can be 0.4:1 to 0.55:1, or at least any one of, equal to any one of, or between any two of 0.4:1, 0.41:1, 0.42:1, 0.43:1, 0.44:1, 0.45:1, 0.46:1, 0.47:1, 0.48:1, 0.49:1, 0.5:1, 0.51:1, 0.52:1, 0.53:1, 0.54:1, and 0.55:1. In some aspects, the homogeneous catalyst solution can include less than 10 ppm, 9 ppm, 8 ppm, 7 ppm, 6 ppm, 5 ppm, 4 ppm, 3 ppm, 2 ppm or 1 ppm, or 0 ppm of iron (Fe). In some aspects, the homogeneous catalyst solution can include less than 10 ppm, 9 ppm, 8 ppm, 7 ppm, 6 ppm, 5 ppm, 4 ppm, 3 ppm, 2 ppm or 1 ppm, or 0 ppm of sodium (Na). In some aspects, the homogeneous catalyst solution can include less than 10 ppm, 9 ppm, 8 ppm, 7 ppm, 6 ppm, 5 ppm, 4 ppm, 3 ppm, 2 ppm or 1 ppm, or 0 ppm of copper (Cu). In some aspects, the homogeneous catalyst solution can include less than 10 ppm, 9 ppm, 8 ppm, 7 ppm, 6 ppm, 5 ppm, 4 ppm, 3 ppm, 2 ppm or 1 ppm, or 0 ppm of Nickel (Ni).

The Co, Mn, and Br containing aqueous first solution 106 can include i) 3 to 7 wt. % or 4 to 6 wt. % or 4.5 to 5.5 wt. % or at least any one of, equal to any one of, or between any two of 3 wt. %, 3.2 wt. %, 3.4 wt. %, 3.6 wt. %, 3.8 wt. %, 4 wt. %, 4.2 wt. %, 4.4 wt. %, 4.5 wt. %, 4.6 wt. %, 4.8 wt. %, 5 wt. %, 5.2 wt. %, 5.4 wt. %, 5.5 wt. %, 5.6 wt. %, 5.8 wt. %, 6 wt. %, 6.2 wt. %, 6.4 wt. %, 6.6 wt. %, 6.8 wt. %, and 7 wt. %, of Co, ii) 1 to 5 wt. % or 2 to 4 wt. % or 2.5 to 3.5 wt. % or at least any one of, equal to any one of, or between any two of 1 wt. %, 1.2 wt. %, 1.4 wt. %, 1.6 wt. %, 1.8 wt. %, 2 wt. %, 2.2 wt. %, 2.4 wt. %, 2.5 wt. %, 2.6 wt. %, 2.8 wt. %, 3 wt. %, 3.2 wt. %, 3.4 wt. %, 3.5 wt. %, 3.6 wt. %, 3.8 wt. %, 4 wt. %, 4.2 wt. %, 4.4 wt. %, 4.6 wt. %, 4.8 wt. %, and 5 wt. %, of Mn, and iii) 12 to 18 wt. % or 14 to 16 wt. % or 14.5 to 15.5 wt. % or at least any one of, equal to any one of, or between any two of 12 wt. %, 12.2 wt. %, 12.4 wt. %, 12.6 wt. %, 12.8 wt. %, 13 wt. %, 13.2 wt. %, 13.4 wt. %, 13.6 wt. %, 13.8 wt. %, 14 wt. %, 14.2 wt. %, 14.4 wt. %, 14.5 wt. %, 14.6 wt. %, 14.8 wt. %, 15 wt. %, 15.2 wt. %, 15.4 wt. %, 15.5 wt. %, 15.6 wt. %, 15.8 wt. %, 16 wt. %, 16.2 wt. %, 16.4 wt. %, 16.6 wt. %, 16.8 wt. %, 17 wt. %, 17.2 wt. %, 17.4 wt. %, 17.6 wt. %, 17.8 wt. %, and 18 wt. %, of Br. The wt. % of Co in the solution 106 can be greater than the wt. % of Mn in the solution 106. The wt. % of Br in the solution 106 can be greater than the wt. % of Co in the solution 106. In some particular aspects, the solution 106 can include 4.5 to 5.5 wt. % of Co, 2.5 to 3.5 wt. % of Mn and 14.4 to 15.4 wt. % of Br. In some aspects, the solution 106 can include an acid. In some aspects, the acid can be acetic acid. In some aspects, the solution 106 can include 10 wt. % to 15 wt. % or at least any one of, equal to any one of, or between any two of 10 wt. %, 11 wt. %, 12 wt. %, 13 wt. %, 14 wt. %, and 15 wt. % of acetic acid. The solution 106 can include a dissolved Co compound e.g., salt. Co in the solution 106 can be present as a Co ion. At least a portion of the Co ion in the solution 106 can be Co⁺². The solution 106 can include a dissolved Mn compound e.g., salt. Mn in the solution 106 can be present as a Mn ion. At least a portion of the Mn ion in the solution 106 can be Mn⁺². The solution 106 can include a dissolved Br compound e.g., salt. At least a portion of Br in the solution 106 can be present as bromide ion (Br−).

The solution 106 can be fed to the feed mix drum 102 at a flow rate of 70 Kg/hr to 90 Kg/hr, or at least any one of, equal to any one of, or between any two of 70 Kg/hr, 71 Kg/hr, 72 Kg/hr, 73 Kg/hr, 74 Kg/hr, 75 Kg/hr, 76 Kg/hr, 77 Kg/hr, 78 Kg/hr, 79 Kg/hr, 80 Kg/hr, 81 Kg/hr, 82 Kg/hr, 83 Kg/hr, 84 Kg/hr, 85 Kg/hr, 86 Kg/hr, 87 Kg/hr, 88 Kg/hr, 89 Kg/hr, and 90 Kg/hr.

In some aspects, the second solution 108 can include 97 wt. % to 100 wt. % or at least any one of, equal to any one of, or between any two of 97 wt. %, 98 wt. %, 99 wt. %, 99.1 wt. %, 99.2 wt. %, 99.3 wt. %, 99.4 wt. %, 99.5 wt. %, 99.6 wt. %, 99.7 wt. %, 99.8 wt. %, 99.9 wt. %, and 100 wt. % of p-xylene. In some aspects, the second solution 108 can include about 99.7 wt. % of p-xylene. In some aspects, the third solution 110 can include 97 wt. % to 100 wt. % or at least any one of, equal to any one of, or between any two of 97 wt. %, 98 wt. %, 98.5 wt. %, 99 wt. %, 99.1 wt. %, 99.2 wt. %, 99.3 wt. %, 99.4 wt. %, 99.5 wt. %, 99.6 wt. %, 99.7 wt. %, 99.8 wt. %, 99.9 wt. %, and 100 wt. % of acetic acid. In some aspects, the third solution 110 can include about 98.5 wt. % of acetic acid. The homogenous catalyst solution in the feed mix drum 102 can include 15 wt. % to 34. 9 wt. % or at least any one of, equal to any one of, or between any two of 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 34.5 wt. %, and 34.9 wt. %, of p-xylene. The homogenous catalyst solution in the feed mix drum 102 can include 65 wt. % to 84. 9 wt. % or at least any one of, equal to any one of, or between any two of 65 wt. %, 66 wt. %, 67 wt. %, 68 wt. %, 69 wt. %, 70 wt. %, 71 wt. %, 72 wt. %, 73 wt. %, 74 wt. %, 75 wt. %, 76 wt. %, 77 wt. %, 78 wt. %, 79 wt. %, 80 wt. %, 81 wt. %, 82 wt. %, 83 wt. %, 84 wt. %, 84.5 wt. %, and 84.9 wt. %, of acetic acid.

A stream 112 including p-xylene in the homogeneous catalyst solution can be fed to the oxidation reactor 104. In some aspects, the stream 112 can be fed to the oxidation reactor 104 at a flow rate 70 Kg/hr to 90 Kg/hr, or at least any one of, equal to any one of, or between any two of 70 Kg/hr, 71 Kg/hr, 72 Kg/hr, 73 Kg/hr, 74 Kg/hr, 75 Kg/hr, 76 Kg/hr, 77 Kg/hr, 78 Kg/hr, 79 Kg/hr, 80 Kg/hr, 81 Kg/hr, 82 Kg/hr, 83 Kg/hr, 84 Kg/hr, 85 Kg/hr, 86 Kg/hr, 87 Kg/hr, 88 Kg/hr, 89 Kg/hr, and 90 Kg/hr. A gaseous stream 114 including oxygen (O₂) can be fed to oxidation reactor 104. The gaseous stream 114 can include 19 vol. % to 25 vol. %, or at least any one of, equal to any one of, or between any two of 19 vol. %, 19.2 vol. %, 19.4 vol. %, 19.6 vol. %, 19.8 vol. %, 20 vol. %, 20.2 vol. %, 20.4 vol. %, 20.6 vol. %, 20.8 vol. %, 21 vol. %, 21.2 vol. %, 21.4 vol. %, 21.6 vol. %, 21.8 vol. %, 22 vol. %, 22.2 vol. %, 22.4 vol. %, 22.6 vol. %, 22.8 vol. %, 23 vol. %, 23.2 vol. %, 23.4 vol. %, 23.6 vol. %, 23.8 vol. %, 24 vol. %, 24.2 vol. %, 24.4 vol. %, 24.6 vol. %, 24.8 vol. %, and 25 vol. % of O₂. In some aspects, the gaseous stream 114 can be air. In the oxidation reactor 104 the p-xylene can be oxidized by O₂ in presence of the Co, Mn, and Br to form terephthalic acid. The p-xylene oxidation reaction condition can include (1) a temperature of 180° C. to 195° C. or at least any one of, equal to any one of, or between any two of 180° C., 181° C., 182° C., 183° C., 184° C., 185° C., 186° C., 187° C., 187.5° C., 188° C., 188.5° C., 189° C., 189.5° C., 190° C., 190.5° C., 191° C., 192° C., 193° C., 194° C., and 195° C. and/or (2) a pressure 5 bar to 20 bar (500 to 2,000 kilopascals) or 10 bar to 14 bar or at least any one of, equal to any one of, or between any two of 5 bar, 6 bar, 7 bar, 8 bar, 9 bar, 10 bar, 11 bar, 12 bar, 13 bar, 14 bar, 15 bar, 16 bar, 17 bar, 18 bar, 19 bar and 20 bar. A product stream 118 comprising terephthalic acid can be produced in the oxidation reactor 104. Residence time of the reaction mixture in the oxidation reactor 104 can be 0.5 hour (hr) to 2 hr or at least any one of, equal to any one of, or between any two of 0.5 hr, 0.6 hr, 0.7 hr, 0.8 hr, 0.9 hr, 1 hr, 1.1 hr, 1.2 hr, 1.3 hr, 1.4 hr, 1.5 hr, 1.6 hr, 1.7 hr, 1.8 hr, 1.9 hr and 2 hr. The oxidation reactor 104 can have a relatively inert inner surface. In some aspects, the oxidation reactor 104 can be a platinum line reactor. In some aspects, the p-xylene and O₂ can fed to the oxidation reactor 104 at a mole ratio 1:3 to 1:5 or at least any one of, equal to any one of, or between any two of 1:3, 1:31, 1:32, 1:33, 1:34, 1:35, 1:36, 1:37, 1:38, 1:39, 1:4, 1:41, 1:42, 1:43, 1:44, 1:45, 1:46, 1:47, 1:48, 1:49, and 1:5. In some aspects, the p-xylene conversion can be 95% to 100% or at least any one of, equal to any one of, or between any two of 95%, 95.5%, 96%, 96.5%, 97%, 97.5%, 98%, 98.5%, 99%, 99.5%, and 100%. In some aspects, the terephthalic acid yield (e.g., % yield) from the reaction between p-xylene and O₂ can be 90% to 100% or at least any one of, equal to any one of, or between any two of 90%, 91%, 92%, 93%, 94%, 95%, 96%, 97%, 98%, 99% and 100%. In some aspects, the terephthalic acid selectivity from the reaction between p-xylene and O₂ can be 98% or higher, preferably 99% or higher, more preferably higher than 99.6% or at least any one of, equal to any one of, or between any two of 98%, 98.2%, 98.4%, 98.6%, 98.8%, 99%, 99.1%, 99.2%, 99.3%, 99.4%, 99.5% 99.6% 99.7% 99.8% 99.9% and 100%. Highly pure, terephthalic acid e.g., crude terephthalic acid can be obtained with the methods and compositions of the present disclosure, crude terephthalic acid refers to the terephthalic acid obtained by the reaction of p-xylene and O_{2,} prior to further purification and/or separation. The product stream 118 can include 97 wt. % to 100 wt. %, or at least any one of, equal to any one of, or between any two of 97 wt. %, 97.5 wt. %, 98 wt. %, 98.5 wt. % 99 wt. %, 99.1 wt. %, 99.2 wt. %, 99.3 wt. %, 99.4 wt. %, 99.5 wt. %, 99.6 wt. %, 99.7 wt. %, 99.8 wt. %, 99.9 wt. % and 100 wt. % of terephthalic acid. 4-carboxybenzaldehyde, p-toluic acid, and/or CO₂ can be formed as side products during p-xylene to terephthalic acid oxidation. The compositions, processes and methods of the present disclosure produce relatively lower amounts of 4-carboxybenzaldehyde, p-toluic acid, and CO₂ (compared to other conventional methods/processes). The product stream 118 can include less than 1 wt. %, or less than 0.5 wt. %, or less than 0.4 wt. %, or less than 0.35 wt. %, or less than 0.3 wt. %, or less than 0.2 wt. %, or 0.1 wt. % or less, or 0.1 wt. % to 0.35 wt. % of 4-carboxybenzaldehyde. The product stream 118 can include less than 1000 ppm, or less than 800 ppm, or less than 700 ppm, or less than 600 ppm, or less than 500 ppm, or less than 400 ppm, or 300 ppm or less, or 300 ppm to 800 ppm of p-toluic acid. CO₂ vol. % in a gaseous effluent stream 116 from the oxidation reactor 104 can be less than 5 vol. %, or less than 4 vol. %, or less than 3 vol. %, or less than 2 vol. %, or less than 1.5 vol. %, or less than 1.4 vol. %, or less than 1.3 vol. %, or less than 1.2 vol. %, or less than 1.1 vol. %, or less than 1 vol. %, or 0.5 vol. % or less, or 0.5 vol. % to 1.5 vol. % of CO₂. A mixture 118 of the product stream having the terephthalic acid and the used CMB catalyst composition can exit the oxidation reactor. In some aspects, the used CMB catalyst composition can be separated from the product stream and recycled to the feed mix drum 102 (not shown). In some aspects, the terephthalic acid can be separated from the used CMB catalyst composition by crystallization of the terephthalic acid. The mother liquor after terephthalic acid crystallization can include used CMB catalyst and can be recycled to the feed mix drum 102. In some aspects, the crystallized terephthalic acid can be dried. In some aspects, stream 118 can be the product stream having the terephthalic acid without the used CMB catalyst composition.

The systems, methods and processes described herein can also include various equipment that is not shown and/or is known to one of skill in the art. For example and without limitation, some controllers, piping, computers, valves, pumps, heaters, thermocouples, pressure indicators, mixers, heat exchangers, and the like may not be shown.

As part of the present disclosure, some specific examples are included below. The examples are for illustrative purposes only and are not intended to limit the present disclosure. Those of ordinary skill in the art will readily recognize parameters that can be changed or modified to yield similar results.

### EXAMPLE

Producing Terephthalic Acid by Oxidation of p-Xylene Using CMB Catalyst

Methods: p-xylene oxidation to terephthalic acid was carried out with air as oxidant, acetic acid solution as solvent, and Co, Mn and Br (CMB) as a catalyst. A reaction mixture, e.g., a homogenous catalyst solution or mixture was obtained by mixing p-xylene, acetic acid solution, and a solution containing Co, Mn and Br. Two parallel experiments were performed, the reaction mixture of a first experiment (experiment 1) included 400 ppm Co, 220 ppm Mn, and 460 ppm Br. The reaction mixture of a second experiment (comparative experiment) included 560 ppm Co, 468 ppm Mn, and 620 ppm Br. The reaction mixture of experiment 1 and the comparative experiment each included about 20 wt. % to 23 wt. % of p-xylene and about 70 wt. % to 75 wt. % of acetic acid. The Co, Mn and Br solution flow rate for experiment 1 was 81 kg/hr and that for the comparative experiment was 101 kg/hr. The reaction mixture for experiment 1 and the comparative experiment was separately contacted with air at a temperature of 189° C. and a pressure 12 bar (1,200 kilopascals) to oxidize p-xylene by oxygen and form terephthalic acid.

Results: Crude terephthalic acid (CTA) yield, specific consumption of p-xylene and acetic acid (AA), and 4-carboxybenzaldehyde (4-CBA) formed in experiment 1 and the comparative experiment was measured. Table 1 shows compositions and methods of the present disclosure results in reduction in 4-CBA formation, increase in CTA yield, and reduction in specific consumption of p-xylene and acetic acid.

TABLE 1
Difference between Experiment 1 and the comparative experiment

| | Performance benefit |
|---|---|
| | experiment 1 over the |
| | comparative experiment |
| CTA Yield (wt. %) | +0.05 |
| 4-CBA (wt. %) | −0.04 |
| Co, Mn and Br solution flow (kg/hr) | −20 |
| Br concentration (%) | −25 |
| Total metals (%) | −40 |
| Specific consumption | −1.00 |
| of p-xylene and AA | |
| (Kg/MetricTon) | |

In Table 1, “+” denotes an increase and “−” denotes a decrease, in experiment 1 over the comparative experiment.

Although embodiments of the present disclosure and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

This disclosure further encompasses the following aspects.

Aspect 1. A process for producing terephthalic acid, the process comprising contacting p-xylene with a gaseous stream comprising oxygen (O₂) in the presence of a homogeneous catalyst solution, at a reaction temperature of 180° C. to 195° C. to oxidize at least a portion of the p-xylene and form a product stream comprising terephthalic acid, the homogeneous catalyst solution comprising 350 ppm to 450 ppm cobalt (Co), 170 ppm to 270 ppm manganese (Mn), and 410 ppm to 510 ppm bromine (Br), wherein a Br/(Co+Mn) wt. % ratio is 0.5:1 to 1:1, and a Co to Mn wt. % ratio is 1.5:1 to 2:1.

Aspect 2. The process of Aspect 1, wherein the homogeneous catalyst solution comprises 390 ppm to 410 ppm Co, 210 ppm to 230 ppm Mn, and 450 ppm to 470 ppm Br.

Aspect 3. The process of any one of Aspects 1 or 2, wherein the Br/(Co+Mn) wt. % ratio is 0.6:1 to 0.9:1, and the Co to Mn wt. % ratio is 1.75:1 to 1.85:1 in the homogeneous catalyst solution.

Aspect 4. The process of any one of Aspects 1 to 3, wherein the reaction temperature is 187° C. to 191° C.

Aspect 5. The process of any one of Aspects 1 to 4, wherein the product stream comprises less than 0.3 wt. % of 4-carboxybenzaldehyde, based on the total weight of the product stream.

Aspect 6. The process of any one of Aspects 1 to 5, wherein the homogeneous catalyst solution further comprises an acid, such as acetic acid.

Aspect 7. The process of any one of Aspects 1 to 6, wherein the gaseous stream comprises 19 vol. % to 25 vol. % of O₂, based on the total volume of the gaseous stream.

Aspect 8. The process of any one of Aspects 1 to 7, wherein the gaseous stream comprises air.

Aspect 9. The process of any one of Aspects 1 to 8, wherein the p-xylene is contacted with the gaseous stream at a reaction pressure of 10 bar to 14 bar (1,000 to 1,400 kilopascals).

Aspect 10. The process of any one of Aspects 1 to 9, wherein the homogeneous catalyst solution is obtained by contacting p-xylene with an aqueous solution comprising 3 to 7 wt. % of Co, 1 to 5 wt. % of Mn and 12 to 18 wt. % of Br.

Aspect 11. The process of Aspect 10, wherein the p-xylene in the homogeneous catalyst solution is contacted with the gaseous stream comprising O₂ in a reactor to form the terephthalic acid and the homogeneous catalyst solution is fed to the reactor at a flow rate 70 Kg/hr to 90 Kg/hr.

Aspect 12. The process of Aspect 11, wherein the reactor is a titanium lined reactor.

Aspect 13. The process of any one of Aspects 11 to 12, wherein the average residence time of the homogeneous catalyst solution in the reactor is 0.5 hours to 2 hours.

Aspect 14. The process of any one of Aspects 11 to 13, wherein a CO₂ vol. % in a gaseous outlet stream of the reactor is less than 1.1 vol. %.

Aspect 15. The process of any one of Aspects 11 to 14, wherein the p-xylene and O₂ is fed to the reactor at a mole ratio 1:3 to 1:5.

Aspect 16. The process of any one of Aspects 1 to 15, wherein the homogeneous catalyst solution is substantially free of or free of iron, sodium, copper, or nickel.

Aspect 17. The process of any one of Aspects 1 to 15, wherein an amount of iron, sodium, copper or nickel in the homogeneous catalyst solution is less than 10 ppm, or is less than 5 ppm.

Aspect 18. The process of any one of Aspects 1 to 17, wherein p-xylene conversion is 95% to 100%.

Aspect 19. The process of any one of Aspects 1 to 18, wherein terephthalic acid yield is 90% to 100%.

Aspect 20. An aqueous solution comprising 3 to 7 wt. % of Co, 1 to 5 wt. % of Mn and 12 to 18 wt. % of Br.

Aspect 21. The aqueous solution of Aspect 20, comprising 4.5 to 5.5 wt. % of Co, 2.5 to 3.5 wt. % of Mn and 14.5 wt. % to 15.5 wt. % of Br.

Aspect 22. The aqueous solution of Aspect 20 or 21, wherein the aqueous solution further comprises dissolved acetic acid.

Aspect 23. The aqueous solution of Aspect 22, wherein the aqueous solution comprises 10 wt. % to 15 wt. % of the acetic acid.

Aspect 24. The aqueous solution of any one of Aspects 20 to 23, wherein at least a portion of the Co in the aqueous solution is present as dissolved Co⁺² ion, at least a portion of the Mn in the solution is present as dissolved Mn⁺² ion, and at least a portion of Br in the aqueous solution is present as dissolved Br⁻ ion.

Aspect 25. The aqueous solution of any one of Aspects 20 to 24, wherein the aqueous solution is substantially free of or free of iron, sodium, copper, or nickel.

The compositions, methods, and articles can alternatively comprise, consist of, or consist essentially of, any appropriate components or steps herein disclosed. The compositions, methods, and articles can additionally, or alternatively, be formulated so as to be devoid, or substantially free, of any steps, components, materials, ingredients, adjuvants, or species that are otherwise not necessary to the achievement of the function or objectives of the compositions, methods, and articles.

The singular forms “a,” “an,” and “the” include plural referents unless the context clearly dictates otherwise. “Or” means “and/or” unless clearly indicated otherwise by context. The terms “first,” “second,” and the like, “primary,” “secondary,” and the like, as used herein do not denote any order, quantity, or importance, but rather are used to distinguish one element from another.

The endpoints of all ranges directed to the same component or property are inclusive and independently combinable (e.g., ranges of “less than or equal to 25 wt %, or 5 to 20 wt %,” is inclusive of the endpoints and all intermediate values of the ranges of “5 to 25 wt %,” etc.). Disclosure of a narrower range or more specific group in addition to a broader range is not a disclaimer of the broader range or larger group.

The suffix “(s)” is intended to include both the singular and the plural of the term that it modifies, thereby including at least one of that term (e.g., the colorant(s) includes at least one colorants). Unless defined otherwise, technical and scientific terms used herein have the same meaning as is commonly understood by one of skill in the art to which this disclosure belongs. A “combination” is inclusive of blends, mixtures, alloys, reaction products, and the like. Unless specified to the contrary herein, a total weight is 100 wt %.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All references are incorporated herein by reference in their entirety.

While typical embodiments have been set forth for the purpose of illustration, the foregoing descriptions should not be deemed to be a limitation on the scope herein. Accordingly, various modifications, adaptations, and alternatives can occur to one skilled in the art without departing from the disclosure herein.

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

In the claims:

1. A process for producing terephthalic acid, the process comprising:
contacting p-xylene with a gaseous stream comprising oxygen (O₂) in the presence of a homogeneous catalyst solution, at a reaction temperature of 180° C. to 195° C. to oxidize at least a portion of the p-xylene and form a product stream comprising terephthalic acid, the homogeneous catalyst solution comprising 350 ppm to 450 ppm cobalt (Co), 170 ppm to 270 ppm manganese (Mn), and 410 ppm to 510 ppm bromine (Br), wherein a Br/(Co+Mn) wt. % ratio is 0.5:1 to 1:1, and a Co to Mn wt. % ratio is 1.5:1 to 2:1.

2. The process of claim 1, wherein the homogeneous catalyst solution comprises 390 ppm to 410 ppm Co, 210 ppm to 230 ppm Mn, and 450 ppm to 470 ppm Br.

3. The process of claim 1, wherein the Br/(Co+Mn) wt. % ratio is 0.6:1 to 0.9:1, and the Co to Mn wt. % ratio is 1.75:1 to 1.85:1 in the homogeneous catalyst solution.

4. The process of claim 1, wherein the reaction temperature is 187° C. to 191° C.

5. The process of claim 1, wherein the product stream comprises less than 0.3 wt. % of 4-carboxybenzaldehyde, based on the total weight of the product stream.

6. The process of claim 1, wherein the homogeneous catalyst solution further comprises an acid.

7. The process of claim 1, wherein the gaseous stream comprises 19 vol. % to 25 vol. % of O₂ based on the total volume of the gaseous stream.

8. The process of claim 1, wherein the gaseous stream comprises air.

9. The process of claim 1, wherein p-xylene is contacted with the gaseous stream at a reaction pressure of 10 bar to 14 bar.

10. The process of claim 1, wherein the homogeneous catalyst solution is obtained by contacting p-xylene with an aqueous solution comprising 3 to 7 wt. % of Co, 1 to 5 wt. % of Mn and 12 to 18 wt. % of Br.

11. The process of claim 10, wherein p-xylene in the homogeneous catalyst solution is contacted with the gaseous stream comprising O₂ in a reactor to form the terephthalic acid and the homogeneous catalyst solution is fed to the reactor at a flow rate 70 Kg/hr to 90 Kg/hr.

12. The process of claim 11, wherein the reactor is a titanium lined reactor.

13. The process of claim 11, wherein the average residence time of the homogeneous catalyst solution in the reactor is 0.5 hours to 2 hours.

14. The process of claim 11, wherein a CO₂ vol. % in a gaseous outlet stream of the reactor is less than 1.1 vol. %.

15. The process of claim 11, wherein the p-xylene and O₂ is fed to the reactor at a mole ratio 1:3 to 1:5.

16. The process of claim 1, wherein the homogeneous catalyst solution is substantially free of or free of iron, sodium, copper, or nickel.

17. The process of claim 1, wherein an amount of iron, sodium, copper and/or nickel in the homogeneous catalyst solution is less than 10 ppm, or is less than 5 ppm.

18. The process of claim 1, wherein p-xylene conversion is 95% to 100%.

19. The process of claim 1, wherein terephthalic acid yield is 90% to 100%.

20. An aqueous solution comprising 3 to 7 wt. % of Co, 1 to 5 wt. % of Mn and 12 to 18 wt. % of Br.

21. The aqueous solution of claim 20, comprising 4.5 to 5.5 wt. % of Co, 2.5 to 3.5 wt. % of Mn and 14.5 wt. % to 15.5 wt. % of Br.

22. The aqueous solution of claim 20, wherein the aqueous solution further comprises dissolved acetic acid.

23. The aqueous solution of claim 22, wherein the aqueous solution comprises 10 wt. % to 15 wt. % of the acetic acid.

24. The aqueous solution of any one of claim 20, wherein at least a portion of the Co in the aqueous solution is present as dissolved Co⁺² ion, at least a portion of the Mn in the solution is present as dissolved Mn⁺² ion, and at least a portion of Br in the aqueous solution is present as dissolved Br⁻ ion.

25. The aqueous solution of any one of claim 20, wherein the aqueous solution is substantially free of or free of iron, sodium, copper, or nickel.
